(19) United States
(12) Patent Application Publication
(10) Pub. No.: US 2019/0183673 A1
(43) Pub. Date: Jun. 20, 2019

(54) COLD COMPRESSION DEVICE

(71) Applicant: Gary CHIU, Plano, TX (US)

(72) Inventor: Gary CHIU, Plano, TX (US)

(21) Appl. No.: 15/849,024

(22) Filed: Dec. 20, 2017

(51) Int. Cl. A61F 7/10; A61H 39/06

(52) U.S. Cl. CPC A61F 7/10; A61F 2007/0277; A61H 39/06

(57) ABSTRACT

A cold compression device has a driving assembly, a tank, a wrap assembly, and a tube assembly. The driving assembly includes a water pump and an air pump. The wrap assembly has a wrap, a water pack in the wrap, and an air pack in the wrap and overlapped on the water pack. The tube assembly has a first tube, a second tube, a third tube, and a fourth tube. The first tube connects the tank and the water pump. The second tube connects the water pump and the water pack. The third tube communicates with the water pack and the tank. The fourth tube connects the air pump and the air pack. Thus, as the first tube is detachable from and put out of the tank, the water pump may draw air into the water pack, which discharges the water in the water pack thoroughly.

### BACKGROUND OF THE INVENTION

### 1. Field of the Invention

The present invention relates to a device for physiotherapy, especially to a device for cold compression.

### 2. Description of the Prior Arts

A conventional cold compression device has a main body, multiple tubes, and a wrap. The main body comprises a tank for receiving ice and water and a water pump communicating with the tank. The tubes are connected to the main body and the wrap so that the iced water is drawn by the water pump to circulate between the tank and the wrap through the tubes. Therefore, with the wrap covering and tightened on a human body, the iced water may cool the covered part of the human body. However, when the conventional cold compression device is stopped using, the water flowing into the wrap is hard to be discharged thoroughly, so bacteria, mildew, or moss may grow in the wrap. Therefore, the water is polluted and the useful lifetime of the water pump and the wrap may be decreased.

Besides, some of the conventional cold compression devices further each have an air pump so that air is drawn by the air pump to circulate between the tank and the wrap through the tubes. However, a common air pump only can pull air into the wrap till the pressure in the wrap arrives at the maximum and then discharges all air from the wrap. In other words, the pressure in the wrap cannot be adjusted. Therefore, no matter by which means the wrap is tightened on the human body, various parts of the human body tightened by the wrap of the conventional cold compression bear the same pressure. Therefore, when the wrap is tightened on some parts of the human body, that person may feel either over tightened or inadequate pressure.

To overcome the shortcomings, the present invention provides a cold compression device to mitigate or obviate the aforementioned problems.

### SUMMARY OF THE INVENTION

The main objective of the present invention is to provide a cold compression device that can discharge water thoroughly.

The cold compression device has a driving assembly, a tank, a wrap assembly, and a tube assembly. The driving assembly comprises: a water pump and an air pump. The wrap assembly comprises a wrap, a water pack and an air pack. Both the water pack and the air pack are in the wrap. The air pack overlaps on the water pack. The tube assembly has a first tube, a second tube, a third tube, and a fourth tube. One end of the first tube selectively extends into and communicates with the tank and another end of the first tube communicates with the water pump. One end of the second tube communicates with the water pump and another end of the second tube communicates with the water pack. One end of the third tube communicates with the water pack and another end of the third tube communicates with the tank. One end of the fourth tube selectively communicates with the air pump and another end of the fourth tube communicates with the air pack.

Therefore, as the first tube is detachable from and put out of the tank, the water pump may draw air into the water pack, which discharges the water in the water pack thoroughly.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a cold compression device in accordance with the present invention;

FIG. 2 is a top view of a second portion of a bag of the cold compression device in FIG. 1;

FIG. 3 is a sectional view of the cold compression device in FIG. 1;

FIG. 4 is a top view of the cold compression device in FIG. 1, showing an opening of a first portion of the bag is opened;

FIG. 5 is a side view of the bag of the cold compression device in FIG. 1, showing the second portion is detached from the first portion;

FIG. 6 is a perspective view of a driving assembly of the cold compression device in FIG. 1; and

FIG. 7 is an exploded view of a wrap assembly of the cold compression device in FIG. 1.

### DETAILED DESCRIPTION OF THE PREFERRED EMBODIMENTS

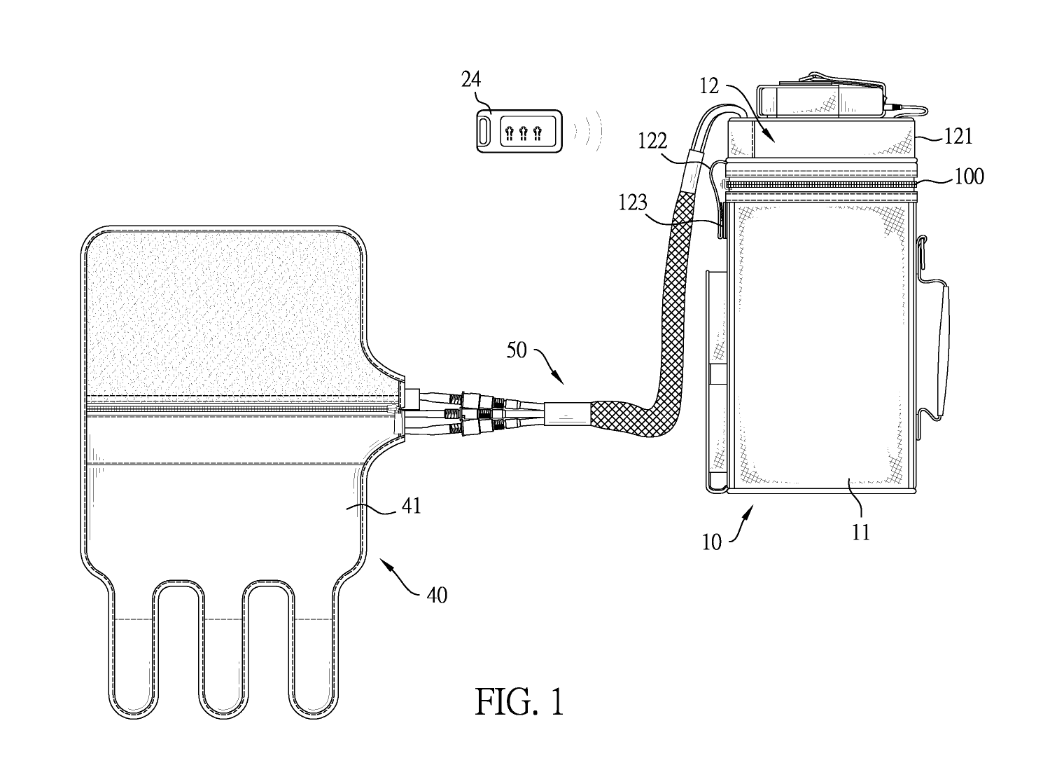

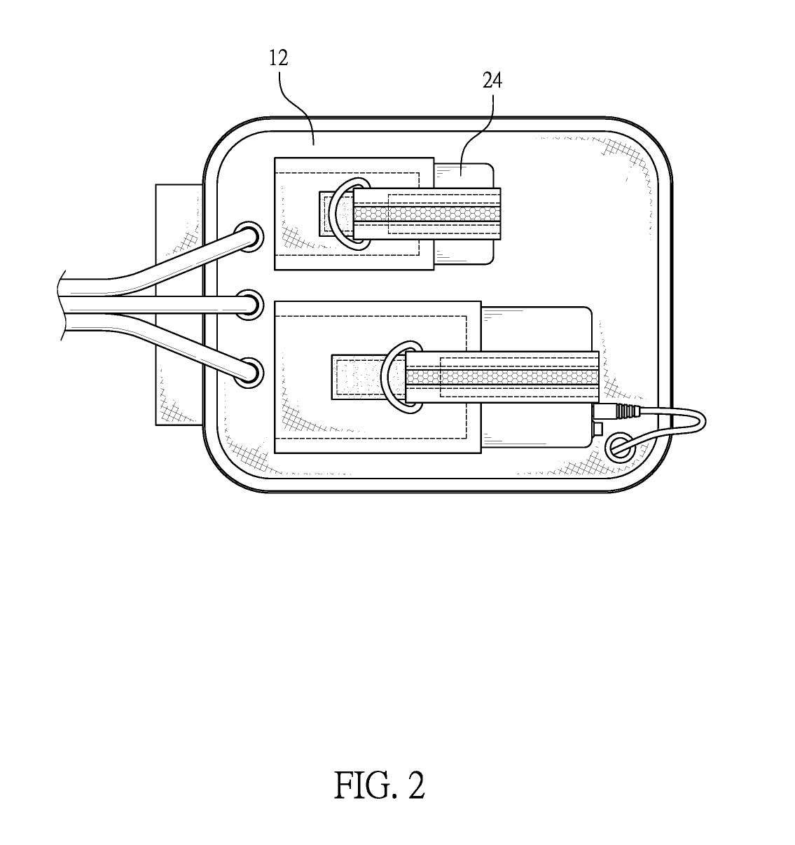

Please refer to FIGS. 1 to 3. A cold compression device in accordance with the present invention is provided. The cold compression device comprises a bag 10, a driving assembly 20, a tank 30, a wrap assembly 40, and a tube assembly 50.

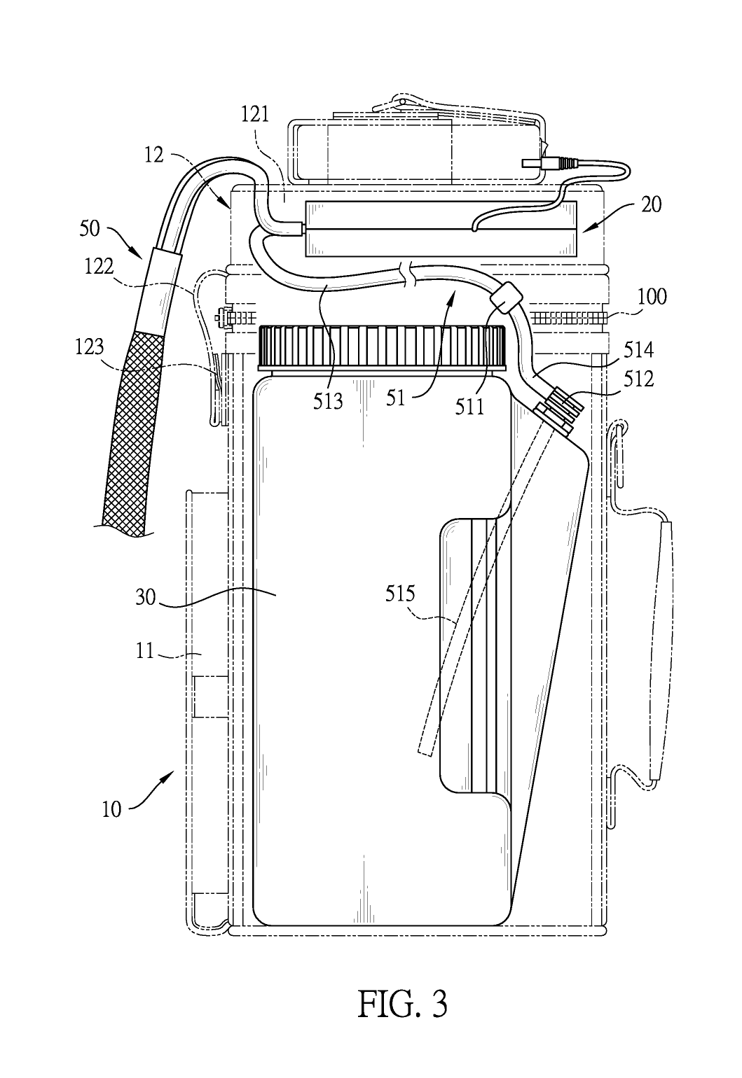

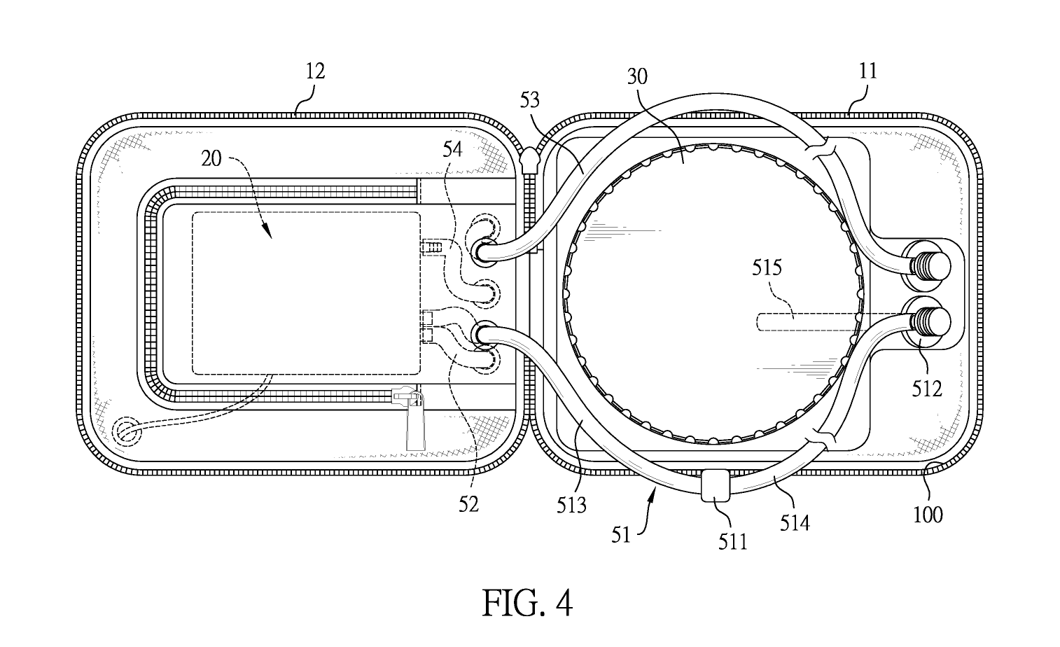

Then please refer to FIGS. 3 to 4. The bag 10 forms an opening 100. The opening 100 is capable of being opened or closed. The bag 10 comprises a first portion 11 and a second portion 12 detachably mounted on the first portion 11. In this embodiment, the opening 100 is formed on the first portion 11, and the second portion 12 selectively closes or opens the opening 100. The second portion 12 is mounted on the first portion 11 by means such as a zipper, button, or buckle.

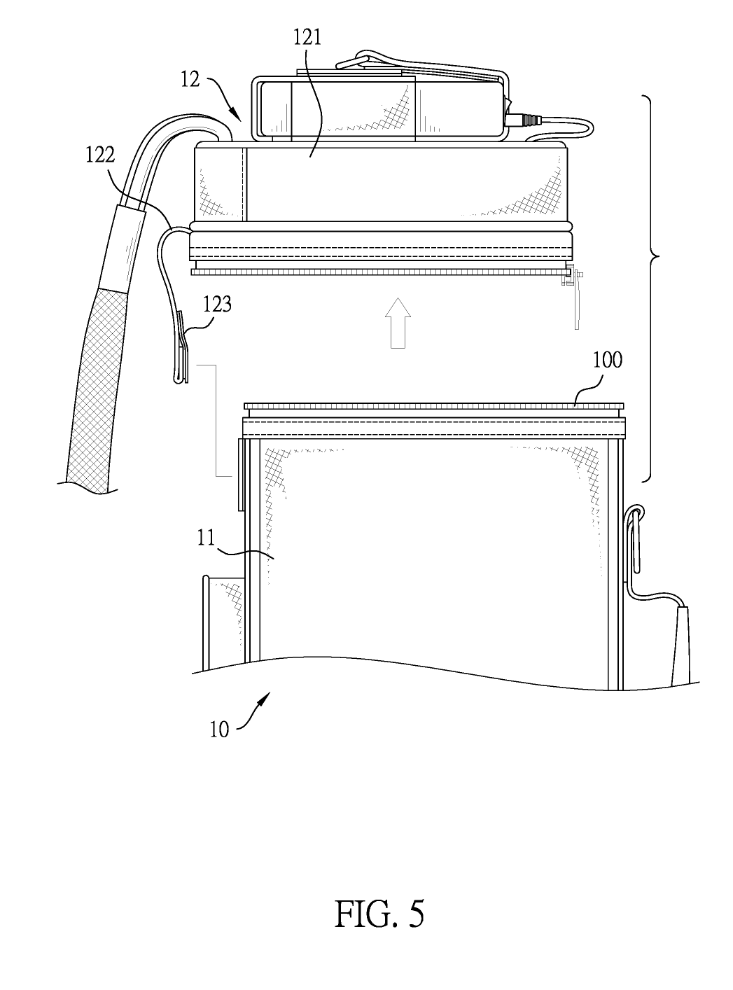

Precisely, in this embodiment, the bag 10 further comprises a zipper arranged on the opening 100, and the second portion 12 comprises a main body 121, a belt 122, and a fastener 123. One end of the belt 122 is securely mounted on the main body 121 of the second portion 12, and the fastener 123 is mounted on another end of the belt 122 so that the belt 122 is detachably mounted with the first portion 11 via the fastener 123. The fastener 123 may be a buckle or a hook and loop fastener. Therefore, when the zipper is opened, the second portion 12 can be moved from the opening 100 and thus the opening 100 is opened (as shown in FIG. 4). However, at this time, the second portion 12 is still connected to the first portion 11 via the belt 122 and the fastener 123 unless the fastener 123 is detached from the first portion 11 (as shown in FIG. 5).

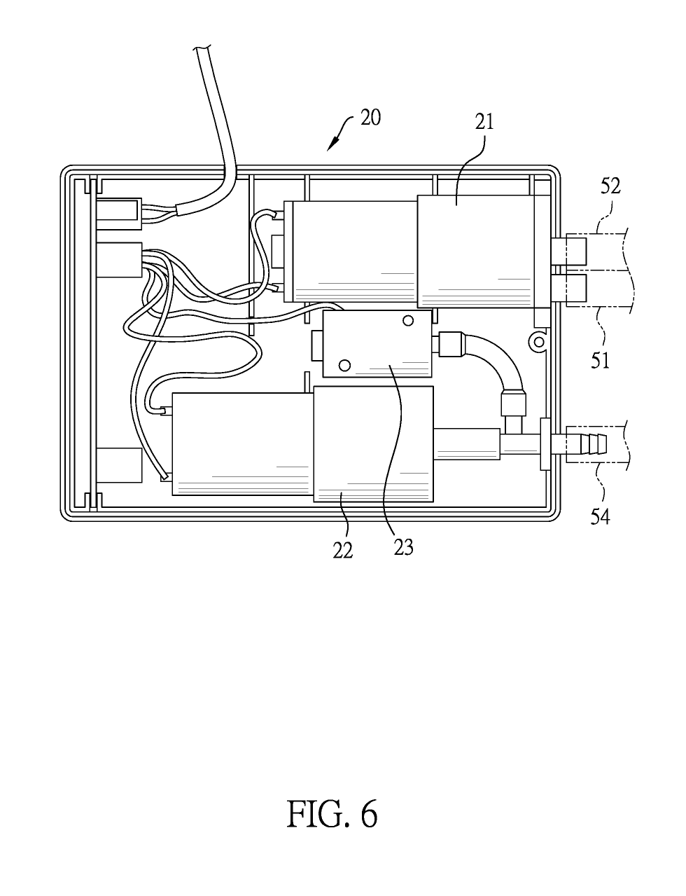

Then please refer to FIGS. 1, 3, and 6. The driving assembly 20 is accommodated in the second portion 12 of the bag 10. The driving assembly 20 comprises a water pump 21, an air pump 22, a valve 23 mounted on the air pump 22, and a controller 24 control the water pump 21 and the air pump 22. In this embodiment, the controller 24 is a remote controller and thus controls the water pump 21 and the air pump 22 via wireless signal, but it is not limited thereto. In another embodiment, the controller 24 may be electrically connected with the water pump 21 and the air pump 22 by a wire.

The tank 30 is accommodated in the first portion 11 of the bag 10 and thus the tank 30 is selectively exposed via the opening 100 of the bag 10 when the opening 100 is open.

The wrap assembly 40 comprises a wrap 41, a water pack 42, and an air pack 43. The water pack 42 and the air pack 43 are disposed in the wrap 41, and the air pack 43 is overlapped on the water pack 42.

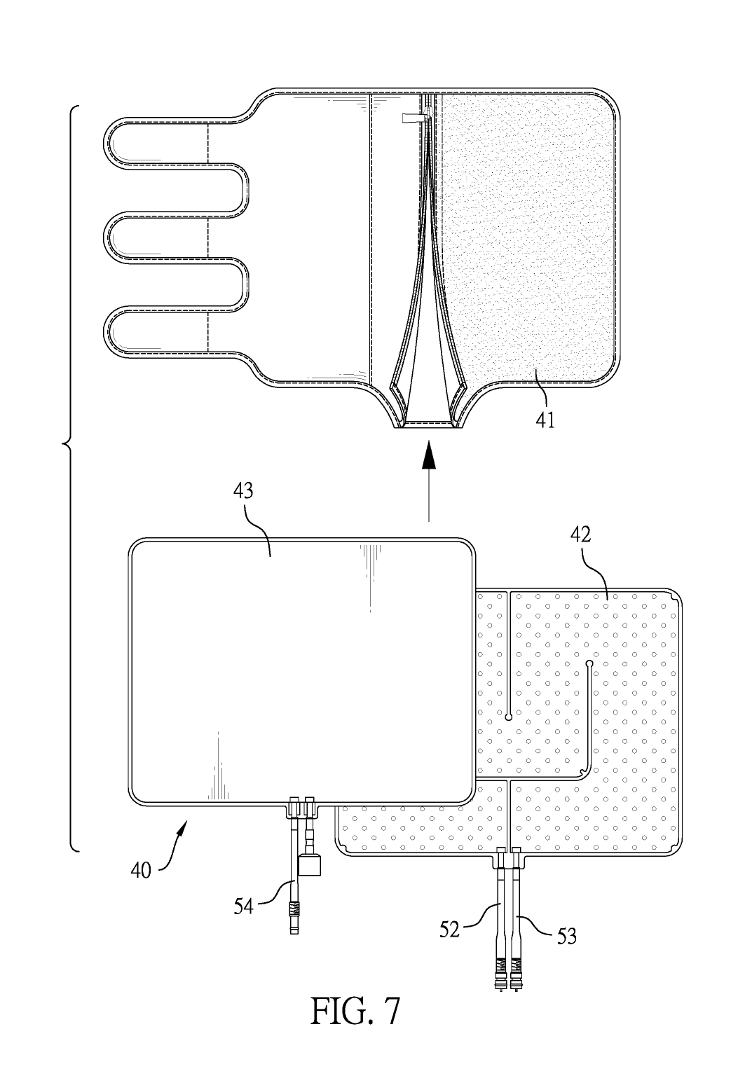

Then please refer to FIGS. 3, 4, and 7. The tube assembly 50 includes a first tube 51, a second tube 52, a third tube 53, and a fourth tube 54.

One end of the first tube 51 selectively extends into and communicates with the tank 30, and another end of the first tube 51 communicates with the water pump 21. Precisely, the first tube 51 comprises a filter component 511, a securing component 512, a first section 513, a second section 514, and a third section 515. The filter component 511 is used for filtering the water flowing in the first tube 51. The securing component 512 is detachably mounted on the tank 30. One end of the first section 513 communicates with the water pump 21 and another end of the first section 513 communicates with the filter component 511. One end of the second section 514 communicates with the filter component 511 and another end of the second section 514 communicates with the securing component 512. One end of the third section 515 communicates with the filter component 511 and another end of the third section 515 extends into and communicates with the tank 30.

One end of the second tube 52 communicates with the water pump 21 and another end of the second tube 52 communicates with the water pack 42. One end of the third tube 53 communicates with the water pack 42 and another end of the third tube 53 detachably communicates with the tank 30. One end of the fourth tube 54 communicates with the air pump 22 and another end of the fourth tube 54 communicates with the air pack 43.

With aforesaid structure, the cold compression device of the present invention can make iced water circulate in the tank 30 and the wrap assembly 40. Precisely, ice and water are put into the tank 30 first, and then the water pump 21 draws the iced water via the first tube 51 and conveys the iced water to flow into the water pack 42 via the second tube 52. As the water pump 21 keeps operating, the iced water is further conveyed back to the tank 30 via the third tube 53.

Therefore, when the wrap assembly 40 is wrapped around a human body, the cold compression device can cold compress the human body. In some circumstances, the user may turn on the air pump 22 to draw air into the air pack 43 via the fourth tube 54, which causes the air pack 43 to expand and thereby the wrap assembly 40 may exert pressure on the human body. Besides, with the valve 23, the user may discharge the air into the air pack 43 at any time. In other words, the pressure exerted on the human body can be adjusted via drawing the air into the air pack 43 by the air pump 22 and discharging the air from the air pack 43 by the valve 23.

After the cold compression is finished, the user can detach the securing component 512 of the first tube 51 from the tank 30, draw the third section 515 of the first tube 51 out of the tank 30, and keep the water pump 21 operating. Therefore, air may be drawn into the water pack 42 via the first tube 51 and the second tube 52 by the water pump 21, and thereby the air may pull the water in the water pack 42 to flow back to the tank 30 via the third tube 53. Therefore, the water may discharge thoroughly from the water pack 42.

Sometimes, the user may only want to press his/her body but does not need cold compressing, so the user can detach the second portion 12 from the first portion 11 of the bag 10 and the first tube 51 and the third tube 53 from the tank 30. After that, the user may turn on the air pump 22 in the second portion 12 of the bag 10 and thus the air is drawn into the wrap assembly 40 for pressing the human body. In other words, the driving assembly 20 and the wrap assembly 40 can work without the tank 30 so that the user does not need to carry the heavy tank.

Consequently, as the first tube 51 can be detached from and put out of the tank 30, the water pump 21 may draw air into the water pack 42, which discharges the water in the water pack 42 thoroughly. Besides, with the air pump 22 comprising the valve 23, the pressure in the air pack 43 can be adjusted through the air pump 22. In addition, the driving assembly 20 and the wrap assembly 40 can work without the tank 30 for only pressing the human body.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A cold compression device comprising:
a driving assembly comprising:
a water pump; and
an air pump;
a tank;
a wrap assembly comprising:
a wrap;
a water pack in the wrap; and
an air pack in the wrap and overlapped on the water pack; and
a tube assembly including:
a first tube, one end of the first tube selectively extending into and communicating with the tank and another end of the first tube communicating with the water pump;
a second tube, one end of the second tube communicating with the water pump and another end of the second tube communicating with the water pack;
a third tube, one end of the third tube communicating with the water pack and another end of the third tube communicating with the tank; and
a fourth tube, one end of the fourth tube communicating with the air pump and another end of the fourth tube communicating with the air pack.

2. The cold compression device as claimed in claim 1, wherein the driving assembly further comprises:
a valve mounted on the air pump.

3. The cold compression device as claimed in claim 1, wherein the driving assembly further comprises:
a controller controlling the water pump and the air pump.

4. The cold compression device as claimed in claim 2, wherein the driving assembly further comprises:
a controller controlling the water pump and the air pump.

5. The cold compression device as claimed in claim 1, wherein the tube assembly further comprises:
a filter component mounted on the first tube.

6. The cold compression device as claimed in claim 4, the tube assembly further comprises:
a filter component mounted on the first tube.

7. The cold compression device as claimed in claim 1, wherein the tube assembly further comprises:
a securing component mounted on the first tube and detachably mounted on the tank.

8. The cold compression device as claimed in claim 5, wherein the tube assembly further comprises:
a securing component mounted on the first tube and detachably mounted on the tank.

9. The cold compression device as claimed in claim 6, wherein the tube assembly further comprises:
a securing component mounted on the first tube and detachably mounted on the tank.

10. The cold compression device as claimed in claim 8, wherein the first tube includes:
a first section; one end of the first section communicating with the water pump and another end of the first section communicating with the filter component;
a second section; one end of the second section communicating with the filter component and another end of the second section communicating with the securing component; and
a third section, one end of the third section communicating with the securing component and another end of the third section selectively extending into and communicating with the tank.

11. The cold compression device as claimed in claim 9, wherein the first tube includes:
a first section; one end of the first section communicating with the water pump and another end of the first section communicating with the filter component;
a second section; one end of the second section communicating with the filter component and another end of the second section communicating with the securing component; and
a third section, one end of the third section communicating with the securing component and another end of the third section selectively extending into and communicating with the tank.

12. The cold compression device as claimed in claim 1 further comprising:
a bag accommodating the driving assembly and the tank.

13. The cold compression device as claimed in claim 11 further comprising:
a bag accommodating the driving assembly and the tank.

14. The cold compression device as claimed in claim 12, wherein the bag forms:
an opening capable of being opened or closed; the tank selectively exposed via the opening when the opening is open.

15. The cold compression device as claimed in claim 13, wherein the bag forms:
an opening capable of being opened or closed; the tank selectively exposed via the opening when the opening is open.

16. The cold compression device as claimed in claim 12, wherein the bag includes:
a first portion accommodating the tank; and
a second portion detachably mounted on the first portion and accommodating the driving assembly;
wherein the first tube and the third tube are detachably mounted on the tank.

17. The cold compression device as claimed in claim 15, wherein the bag includes:
a first portion accommodating the tank; and
a second portion detachably mounted on the first portion and accommodating the driving assembly;
wherein the first tube and the third tube are detachably mounted on the tank.
